(19) United States
(12) Patent Application Publication
(10) Pub. No.: US 2018/0271766 A1
(43) Pub. Date: Sep. 27, 2018

(54) ACRYLIC POLYMER COMPRISING ALKOXYSILANE GROUPS AND COSMETIC USES THEREOF

(71) Applicant: L'OREAL, Paris (FR)

(72) Inventors: Xavier SCHULTZE, Les Pavillons sous Bois (FR); Franck HERNANDEZ, Villemomble (FR); Bertrand LION, Paris (FR); Julien PORTAL, Les Pavillons sous Bois (FR)

(21) Appl. No.: 15/522,864

(22) PCT Filed: Oct. 29, 2015
(86) PCT No.: PCT/EP2015/075124

§ 371 Date: Apr. 28, 2017

(30) Foreign Application Priority Data
Oct. 31, 2014 (FR) 1460490

(51) Int. Cl. A61K 8/58; A61K 8/81; A61Q 19/08; C08L 33/08

(52) U.S. Cl. CPC A61K 8/585; A61K 8/8164; C08K 5/544; C08L 33/08; A61K 2800/95; A61Q 19/08

(57) ABSTRACT

The invention relates to a cosmetic process for caring for or making up keratin materials, comprising either the topical application to the keratin materials of an (extemporaneous) anhydrous mixture of a cosmetic composition comprising a maleic anhydride acrylic polymer and of an amino alkoxysilane compound (I); or the sequential application to the keratin materials of an anhydrous cosmetic composition comprising a maleic anhydride acrylic polymer and of an amino alkoxysilane compound (I), wherein said maleic anhydride acrylic polymer is obtained by polymerization of:

(a) 50% to 90% by weight, relative to the total weight of monomers, of isobornyl (meth)acrylate
(b) 1% to 50% by weight of maleic anhydride
(c) 0% to 49% by weight of additional (meth)acrylate monomer;
said amino alkoxysilane having the formula (I):

R—NH—R₁Si(OR₂)_{z}(R₃)ₓ (I)

in which:
R₁ is a C₁-C₂₀ hydrocarbon-based divalent group,
R=H or a C₁-C₄ alkyl group,
R₂ and R₃ represent an alkyl group comprising from 1 to 6 carbon atoms;
z denotes an integer ranging from 1 to 3, and
x denotes an integer ranging from 0 to 2,
with z+x=3.

Uses: skin-tensioning agent, makeup, hair fixing.

The present invention relates to an acrylic polymer bearing alkoxysilane groups, to a composition comprising such a polymer and to the use of this polymer in the cosmetic field.

During the aging process, various signs appear on the skin, which are very characteristic of this aging, resulting in particular in a modification of skin structure and functions. The main clinical signs of skin aging are in particular the appearance of fine lines and deep wrinkles, which increase with age.

It is known practice to treat these signs of aging using cosmetic or dermatological compositions containing active agents capable of combating aging, such as α-hydroxy acids, β-hydroxy acids and retinoids. These active agents act on wrinkles by eliminating dead skin cells and by accelerating the cell renewal process. However, these active agents have the drawback of only being effective for the treatment of wrinkles after a certain application time. As it happens, it is increasingly sought to obtain an immediate effect of the active agents used, rapidly resulting in smoothing-out of wrinkles and fine lines and in the disappearance of the signs of fatigue.

The inventors have discovered that a particular maleic anhydride acrylic polymer combined with a particular aminosilane has good film-forming properties. When the polymer combined with said aminosilane is applied to the skin, it has a good tensioning effect on the skin and thus makes it possible to attenuate the wrinkles of the skin, in particular rapidly or immediately after application to the skin. This tensioning effect also exhibits good water resistance, and therefore good persistence with respect to water. This particular acrylic polymer is readily conveyable in a hydrocarbon-based oil such as isododecane. This polymer, combined with said aminosilane, is thus very suitable for preparing anhydrous compositions which have good water resistance and have in particular a good tensioning effect.

This maleic anhydride acrylic polymer combined with said aminosilane forms a film-forming deposit that is also suitable for making up the skin or the lips or the eyelashes, such as foundations, lipsticks or mascaras, or for fixing the hair.

The film-forming deposit obtained has good water resistance. It also has good resistance to oil (especially to olive oil) and to sebum.

More specifically, a subject of the present invention is a process, in particular a cosmetic process, for caring for or making up keratin materials, in particular the skin, more particularly facial skin, in particular wrinkled skin, comprising:

either the topical application to the keratin materials, in particular to the skin, of an anhydrous (extemporaneous) mixture of a cosmetic composition comprising a maleic anhydride acrylic polymer and of an amino alkoxysilane compound (I) or of a cosmetic composition containing same;
or the sequential application to the keratin materials, in particular to the skin, of an anhydrous cosmetic composition comprising a maleic anhydride acrylic polymer and of an amino alkoxysilane compound (I) or of an anhydrous cosmetic composition containing same,
said maleic anhydride acrylic polymer being able to be obtained by polymerization of:
(a) 50% to 90% by weight, relative to the total weight of monomers, of isobornyl (meth)acrylate
(b) 1% to 50% by weight of maleic anhydride
(c) 0 to 49% by weight of additional (meth)acrylate monomer chosen from:
(i) linear or branched, saturated or unsaturated C₁-C₂₀ alkyl (meth)acrylates, optionally interrupted with one or more non-adjacent heteroatoms chosen from O and S or with a group NR, R being a C₁-C₄ alkyl group, optionally substituted with a phenyl or furfuryl group;
(ii) saturated C₄-C₈ cycloalkyl (meth)acrylates optionally interrupted with 0 or NH;
said amino alkoxysilane having the formula (I):

R—NH—R₁Si(OR₂)_{z}(R₃)ₓ (I)

in which:
R₁ is a linear or branched, saturated or unsaturated, cyclic or acyclic C₁-C₂₀ hydrocarbon-based divalent group, which may be interrupted in its chain with a heteroatom (O, S, NH) or a carbonyl group (CO), R₁ being linked to the silicon atom directly via a carbon atom;
R=H or a C₁-C₄ alkyl group, preferably H;
R₂ and R₃, which may be identical or different, represent a linear or branched alkyl group comprising from 1 to 6 carbon atoms,
z denotes an integer ranging from 1 to 3, and
x denotes an integer ranging from 0 to 2,
with z+x=3.

The process according to the invention is suitable for caring for or making up keratin materials, such as the skin, the lips, the eyelashes, the hair or the nails.

The process according to the invention is in particular intended to be carried out for caring for the skin, more particularly facial skin, in particular wrinkled skin.

The process according to the invention is in particular intended for smoothing out human facial and/or body skin and/or for decreasing or effacing the signs of skin aging, in particular for reducing or effacing wrinkles and/or fine lines on the skin.

According to one embodiment of the process according to the invention, an extemporaneous mixture of an anhydrous composition comprising a maleic anhydride acrylic polymer and of an amino alkoxysilane (I), or an anhydrous composition containing same and comprising a physiologically acceptable medium, as defined below, is applied topically to keratin materials.

According to one embodiment of the process according to the invention, an extemporaneous mixture of an anhydrous composition comprising a maleic anhydride acrylic polymer and of an amino alkoxysilane (I), or an anhydrous composition containing same and comprising a physiologically acceptable medium, as defined below, is applied topically to the skin.

According to another embodiment of the process according to the invention, an anhydrous composition comprising an acrylic polymer as described previously and of an amino alkoxysilane (I), or an anhydrous composition containing same and comprising a physiologically acceptable medium, as defined previously, are applied sequentially to keratin materials.

According to another embodiment of the process according to the invention, an anhydrous composition comprising an acrylic polymer as described previously and of an amino alkoxysilane (I), or an anhydrous composition containing same and comprising a physiologically acceptable medium, as defined previously, are applied sequentially to the skin.

A subject of the invention is also the cosmetic use, as a tensioning agent for the skin, in particular for wrinkled skin, of a maleic anhydride acrylic polymer as described previously, as a mixture with an amino alkoxysilane (I), or of an anhydrous composition containing same and comprising a physiologically acceptable medium, as defined below.

A subject of the invention is also a composition, especially a cosmetic composition, obtained by mixing an anhydrous composition comprising said maleic anhydride acrylic polymer and an amino alkoxysilane (I) or an anhydrous composition containing same and comprising a physiologically acceptable medium, as defined below.

A subject of the invention is also a kit comprising a first anhydrous composition comprising said maleic anhydride acrylic polymer as described previously and a second anhydrous composition comprising an amino alkoxysilane (I) as described previously and comprising a physiologically acceptable medium, the first and second compositions each being packaged in a separate packaging assembly.

The composition packaging assembly is, in a known manner, any packaging that is suitable for storing cosmetic compositions (in particular a bottle, tube, spray bottle or aerosol bottle).

Such a kit allows the skin treatment process according to the invention to be performed.

The term “tensioning agent” is intended to mean compounds that are capable of having a noticeable tensioning effect, i.e. of smoothing out the skin and immediately reducing the wrinkles and fine lines, or even making them disappear.

The tensioning effect may be characterized by means of an in vitro retraction test as described in example 5.

The maleic anhydride acrylic polymer used according to the invention comprises an isobornyl (meth)acrylate, maleic anhydride and optionally an additional acrylate monomer as defined previously. Advantageously, the maleic anhydride acrylic polymer is formed essentially from these monomers in the contents described previously.

Advantageously, the polymer used according to the invention is derived from the polymerization of:

(a) 50% to 90% by weight, relative to the total weight of monomers, of isobornyl (meth)acrylate
(b) 5% to 50% by weight of maleic anhydride
(c) 0 to 30% by weight of additional (meth)acrylate monomer as described previously.

Advantageously, the polymer used according to the invention is derived from the polymerization of:

(a) 50% to 80% by weight, relative to the total weight of monomers, of isobornyl (meth)acrylate
(b) 5% to 30% by weight of maleic anhydride
(c) 15 to 30% by weight of additional (meth)acrylate monomer as described previously.

Advantageously, the polymer used according to the invention is derived from the polymerization of:

(a) 50% to 80% by weight, relative to the total weight of monomers, of isobornyl (meth)acrylate
(b) 5% to 25% by weight of maleic anhydride
(c) 15 to 30% by weight of additional (meth)acrylate monomer as described previously.

Advantageously, the polymer used according to the invention is derived from the polymerization of:

(a) 50% to 80% by weight, relative to the total weight of monomers, of isobornyl (meth)acrylate
(b) 5% to 15% by weight of maleic anhydride
(c) 15 to 30% by weight of additional (meth)acrylate monomer as described previously.

Advantageously, the polymer used according to the invention is derived from the polymerization of:

(a) 60% to 80% by weight, relative to the total weight of monomers, of isobornyl (meth)acrylate
(b) 5% to 12% by weight of maleic anhydride
(c) 15 to 30% by weight of additional (meth)acrylate monomer as described previously.

Advantageously, the polymer used according to the invention is derived from the polymerization of:

(a) 60% to 80% by weight, relative to the total weight of monomers, of isobornyl (meth)acrylate
(b) 5% to 12% by weight of maleic anhydride
(c) 18 to 30% by weight of additional (meth)acrylate monomer as described previously.

The additional (meth)acrylate monomer is preferably chosen from C₆-C₁₆ alkyl (meth)acrylates, and preferentially chosen from C₆-C₁₆ alkyl acrylates.

As examples of C₆-C₁₆ alkyl (meth)acrylates, mention may be made of hexyl (meth)acrylate, octyl (meth)acrylate, 2-ethylhexyl (meth)acrylate, decyl (meth)acrylate and lauryl (meth)acrylate. 2-Ethylhexyl acrylate is preferably used.

Preferably, the polymer used according to the invention comprises, or consists of, isobornyl acrylate, 2-ethylhexyl acrylate and maleic anhydride.

A particularly preferred polymer used according to the invention is derived from the polymerization of:

(a) 50% to 80% by weight, relative to the total weight of monomers, of isobornyl (meth)acrylate
(b) 5% to 30% by weight of maleic anhydride
(c) 15% to 30% by weight of C₆-C₁₆ alkyl acrylate monomer.

A particularly preferred polymer used according to the invention is derived from the polymerization of:

(a) 50% to 80% by weight, relative to the total weight of monomers, of isobornyl (meth)acrylate
(b) 5% to 25% by weight of maleic anhydride
(c) 15% to 30% by weight of C₆-C₁₆ alkyl acrylate monomer.

A particularly preferred polymer used according to the invention is derived from the polymerization of:

(a) 50% to 80% by weight, relative to the total weight of monomers, of isobornyl (meth)acrylate
(b) 5% to 15% by weight of maleic anhydride
(c) 15% to 30% by weight of C₆-C₁₆ alkyl acrylate monomer.

A particularly preferred polymer used according to the invention is derived from the polymerization of:

(a) 60% to 80% by weight, relative to the total weight of monomers, of isobornyl (meth)acrylate
(b) 5% to 12% by weight of maleic anhydride
(c) 15% to 30% by weight of C₆-C₁₆ alkyl acrylate monomer.

A particularly preferred polymer used according to the invention is derived from the polymerization of:

(a) 60% to 80% by weight, relative to the total weight of monomers, of isobornyl (meth)acrylate
(b) 5% to 12% by weight of maleic anhydride
(c) 18% to 30% by weight of C₆-C₁₆ alkyl acrylate monomer.

Advantageously, the polymer used according to the invention consists of the monomers described previously.

Advantageously, the polymer used according to the invention is nonionic.

Preferably, the maleic anhydride acrylic polymer used according to the invention has a weight-average molecular weight ranging from 5000 to 1 000 000 g/mol, preferably ranging from 10 000 to 500 000 g/mol and preferentially ranging from 15 000 to 350 000 g/mol.

The molecular weight may especially be determined by steric exclusion chromatography, with THF eluent, polystyrene standard, 2414 refractometric detector from Waters.

The copolymer may be a random, alternating (block) or gradient polymer. Preferably, the copolymer is random.

The copolymer used according to the invention may be prepared by radical polymerization of the monomers described previously, especially as a mixture or added sequentially during the polymerization, especially using an organic solvent with a boiling point of greater than or equal to 60° C., for instance isododecane, ethanol, ethyl acetate, tetrahydrofuran, methyltetrahydrofuran or methyl ethyl ketone. The organic solvent makes it possible to dissolve the monomers used and the polymer formed.

The polymerization is in particular performed in the presence of a radical initiator in particular of peroxide type (for example tert-butyl peroxy-2-ethylhexanoate: Trigonox 21S; 2,5-dimethyl-2,5-bis(2-ethylhexanoylperoxy)hexane: Trigonox 141; tert-butyl peroxypivalate: Trigonox 25C75 from AkzoNobel) or of azo type, for example (AIBN: azobisisobutyronitrile; V50: 2,2′-azobis(2-amidinopropane) dihydrochloride).

The polymerization may be performed at a temperature ranging from 60 to 100° C., and preferably ranging from 60 to 85° C.

The polymerization time may be about 24 hours.

The polymer used according to the invention may be used in an anhydrous composition comprising a physiologically acceptable medium, in particular in a cosmetic composition.

The term “physiologically acceptable medium” means a medium that is compatible with human keratin materials and in particular with the skin.

The term “cosmetic composition” is understood to mean a composition that is compatible with keratin materials, which has a pleasant color, odor and feel and which does not cause unacceptable discomfort (stinging, tautness or redness) liable to discourage the consumer from using it.

The maleic anhydride acrylic polymer as defined previously may be present in the composition according to the invention in a content ranging from 0.1% to 10% by weight, relative to the total weight of the composition, preferably from 0.5% to 10% by weight of active material, preferentially ranging from 1% to 8% by weight, and more preferentially ranging from 1% to 6% by weight.

For the amino alkoxysilane of formula (I) used in the process according to the invention:

Preferably, R₂ represents an alkyl group comprising from 1 to 4 carbon atoms.

Preferably, R₂ represents a linear alkyl group comprising from 1 to 4 carbon atoms.

Preferably, R₂ represents an ethyl group.

Preferably, R₃ represents an alkyl group comprising from 1 to 4 carbon atoms.

Preferably, R₃ represents a linear alkyl group comprising from 1 to 4 carbon atoms. Preferably, R₃ represents a methyl or ethyl group.

Preferably, R=H.

Preferably, R₁ is an acyclic chain.

Preferably, R₁ is a linear or branched, saturated or unsaturated C₁-C₆ hydrocarbon-based chain. Preferentially, R₁ is a linear saturated C₁-C₆ hydrocarbon-based chain. More preferentially, R₁ is a linear saturated C₂-C₄ hydrocarbon-based chain.

Preferably, R₁ is a substituted linear saturated C₁-C₆ hydrocarbon-based chain,

### R=H,

R₂ represents an alkyl group comprising from 1 to 4 carbon atoms,
R₃ represents an alkyl group comprising from 1 to 4 carbon atoms.

Preferably, z is equal to 3.

Preferably, the amino alkoxysilane of formula (I) is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane.

Preferably, the amino alkoxysilane (I) is chosen from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane.

Preferably, the amino alkoxysilane (I) is 3-aminopropyltriethoxysilane (APTES).

Advantageously, the amino alkoxysilane is used in a mole ratio of amino alkoxysilane/maleic anhydride group of the acrylic polymer ranging from 0.01 to 10, preferably ranging from 0.1 to 5, preferentially ranging from 0.1 to 2 and more preferentially ranging from 0.1 to 1.

The amino alkoxysilane (I) used reacts with the maleic anhydride group present in the polymer to form a unit having the following formula:

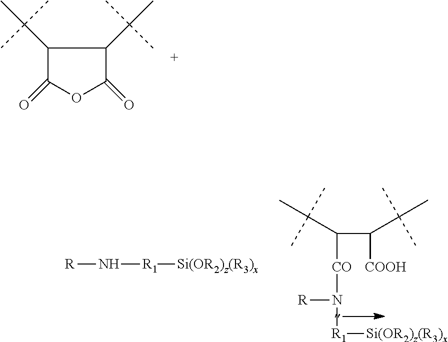

Amide and carboxylic acid unit

Such a polymer bearing an amino alkoxysilane group is novel and thus also forms the subject of the present invention. A subject of the invention is also an anhydrous composition comprising such a polymer bearing an amino alkoxysilane group and a physiologically acceptable medium.

The polymer bearing an amino alkoxysilane group may thus be obtained by reacting the amino alkoxysilane (I) with the maleic anhydride acrylic polymer described previously. Some or all of the anhydride groups react with the NH group of the compound (I) and form a unit bearing an amide group and a carboxylic acid group as described previously.

According to one embodiment of the process according to the invention, a mixture, especially an extemporaneous mixture, of the maleic anhydride acrylic polymer and of an amino alkoxysilane (I) is applied to the keratin materials, in particular to the skin. It is also possible to perform sequential application of, on the one hand, the maleic anhydride acrylic polymer and, on the other hand, an amino alkoxysilane (I) as defined previously.

The composition used according to the invention is generally suitable for topical application to keratin materials, in particular to the skin, and thus generally comprises a physiologically acceptable medium, i.e. a medium that is compatible with the skin and/or its integuments. It is preferably a cosmetically acceptable medium, i.e. a medium which has a pleasant color, odor and feel and which does not cause any unacceptable discomfort (stinging, tautness or redness) liable to discourage the consumer from using this composition.

According to a preferred embodiment of the invention, the composition comprising the maleic anhydride acrylic polymer may contain a hydrocarbon-based oil.

The hydrocarbon-based oil is an oil that is liquid at room temperature (25° C.).

The term “hydrocarbon-based oil” means an oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

The hydrocarbon-based oil may be volatile or nonvolatile.

The hydrocarbon-based oil may be chosen from:

hydrocarbon-based oils containing from 8 to 14 carbon atoms, and especially:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade name Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in examples 1 and 2 of patent application WO 2008/155 059 from the company Cognis, and mixtures thereof,
   short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have chain lengths varying from C₄ to C₂₄, these chains possibly being linear or branched, and saturated or unsaturated; these oils are especially heptanoic or octanoic acid triglycerides, or alternatively wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, rapeseed oil, blackcurrant oil, evening primrose oil, millet oil, barley oil, quinoa oil, rye oil, safflower oil, candlenut oil, passion-flower oil and musk rose oil; shea butter; or else caprylic/capric acid triglycerides, for instance those sold by the company Stéarineries Dubois or those sold under the names Miglyol 810®, 812® and 818® by the company Dynamit Nobel,
synthetic ethers having from 10 to 40 carbon atoms;
linear or branched hydrocarbons of mineral or synthetic origin, such as petroleum jelly, polydecenes, hydrogenated polyisobutene such as Parleam®, squalane and liquid paraffins, and mixtures thereof,
synthetic esters such as oils of formula R₁COOR₂ in which R₁ represents a linear or branched fatty acid residue containing from 1 to 40 carbon atoms and R₂ represents an, in particular branched, hydrocarbon-based chain containing from 1 to 40 carbon atoms, on condition that R₁+R₂≥10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalkyl heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

Advantageously, the hydrocarbon-based oil is apolar (thus formed solely from carbon and hydrogen atoms).

The hydrocarbon-based oil is preferably chosen from hydrocarbon-based oils containing from 8 to 14 carbon atoms, in particular the apolar oils described previously.

Preferentially, the hydrocarbon-based oil is isododecane.

The composition comprising the polymer may contain, in addition to the hydrocarbon-based oil, a silicone oil. The term “silicone oil” means an oil comprising at least one silicon atom and especially at least one Si—O group. The silicone oil may be volatile or nonvolatile.

The term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapor pressure, at room temperature and at atmospheric pressure, in particular having a vapor pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg). The term “nonvolatile oil” means an oil with a vapor pressure of less than 0.13 Pa.

Volatile silicone oils that may be mentioned include volatile linear or cyclic silicone oils, especially those with a viscosity ≤8 centistokes (cSt) (8×10⁻⁶ m²/s), and especially having from 2 to 10 silicon atoms and in particular from 2 to 7 silicon atoms, these silicones optionally comprising alkyl or alkoxy groups having from 1 to 10 carbon atoms. As volatile silicone oil that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As nonvolatile silicone oils, mention may be made of linear or cyclic nonvolatile polydimethylsiloxanes (PDMSs); polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

Advantageously, the composition may comprise a hydrocarbon-based oil in a content ranging from 60% to 100% by weight relative to the total weight of the oils present in the composition and from 0 to 40% by weight of silicone oil. According to a preferred embodiment of the invention, the composition contains as oil only a hydrocarbon-based oil.

The composition according to the invention may comprise a cosmetic additive chosen from fragrances, preserving agents, fillers, UV-screening agents, oils, waxes, surfactants, moisturizers, vitamins, ceramides, antioxidants, free-radical scavengers, polymers, thickeners and colorants.

The composition according to the invention may also comprise a colorant such as pulverulent colorants, liposoluble dyes or water-soluble dyes. This colorant may be present in a content ranging from 0.01% to 30% by weight, relative to the total weight of the composition.

The pulverulent colorants may be chosen from pigments and nacres.

The pigments may be white or colored, mineral and/or organic, and coated or uncoated. Among the mineral pigments that may be mentioned are titanium dioxide, optionally surface-treated, zirconium, zinc or cerium oxides, and also iron or chromium oxides, manganese violet, ultramarine blue, chromium hydrate and ferric blue. Among the organic pigments that may be mentioned are carbon black, pigments of D & C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

Advantageously, the composition according to the invention is a skincare composition.

Advantageously, the composition according to the invention is a skincare composition.

The composition according to the invention may be a makeup composition such as a foundation, a lipstick or an eye liner.

According to one embodiment, the composition according to the invention is a makeup composition and comprises a volatile oil and a nonvolatile oil as described previously. In particular, the makeup composition may comprise a hydrocarbon-based volatile oil and a hydrocarbon-based nonvolatile oil.

According to one embodiment, the composition according to the invention is a hair fixing composition.

According to one embodiment, the composition according to the invention is an anhydrous composition. The term “anhydrous composition” means a composition containing less than 2% by weight of water, or even less than 0.5% of water, and especially free of water. Where appropriate, such small amounts of water may especially be introduced by ingredients of the composition that may contain residual amounts thereof.

Needless to say, those skilled in the art will take care to select this or these optional additional compounds, and/or the amount thereof, such that the anti-wrinkle properties of the composition according to the invention are not, or are not substantially, adversely affected by the envisaged addition.

According to a first embodiment of the process according to the invention, a mixture, especially an extemporaneous mixture, of an anhydrous cosmetic composition comprising the maleic anhydride acrylic polymer and of an amino alkoxysilane (I), as previously described, is applied to the keratin materials, especially to the skin. The extemporaneous mixture is advantageously prepared less than 5 minutes before it is applied to the keratin materials, especially to the skin, and preferably less than 3 minutes.

According to a second embodiment of the process according to the invention, an anhydrous composition, especially a cosmetic anhydrous composition, comprising the acrylic polymer is first applied to the keratin materials, especially to the skin, and an amino alkoxysilane (I) or an anhydrous cosmetic composition containing same is then applied. The application of the amine compound may be performed after a time of between 5 minutes and one hour after having applied the acrylic polymer to the keratin materials, especially to the skin.

According to a third embodiment of the process according to the invention, the amino alkoxysilane (I), or a cosmetic anhydrous composition containing same, is first applied to the keratin materials, especially to the skin, and the anhydrous composition, especially cosmetic anhydrous composition, comprising the acrylic polymer is then applied. The application of the acrylic polymer may be performed after a time of between 5 minutes and one hour after having applied the amino alkoxysilane to the keratin materials, especially to the skin.

The application of the cosmetic composition used according to the invention is performed according to the usual techniques, for example by application (in particular of creams, gels, sera or lotions) to the skin intended to be treated, in particular facial and/or neck skin, especially the skin of the area around the eyes. In the context of this process, the composition may, for example, be a care composition.

The invention will now be described with reference to the following examples, which are given as non-limiting illustrations.

### EXAMPLE 1: ISOBORNYL ACRYLATE/2-ETHYLHEXYL ACRYLATE/MALEIC ANHYDRIDE COPOLYMER (70/20/10 BY WEIGHT)

70 g of isobornyl acrylate, 20 g of 2-ethylhexyl acrylate and 10 g of maleic anhydride were placed in a jacketed 1-liter reactor equipped with a stirring anchor. A mixture of 70 g of isododecane and 30 g of ethyl acetate was then added. The medium was brought to a temperature of 40° C. with stirring (150 rpm) and was sparged with argon for 10 minutes, followed by addition of 0.5 g of initiator tert-butyl peroxy-2-ethylhexanoate Trigonox 21S (Trigonox® 21S from AkzoNobel).

The heating of the jacket was set at 90° C. for 7 hours at 150 rpm.

The medium was then diluted with 300 g of isododecane, and then concentrated by distillation to remove the ethyl acetate and the unreacted maleic anhydride.

A solution containing 30% by weight of the copolymer in isododecane (yield of greater than 90%) was finally obtained.

The polymer obtained has a molecular weight (Mw) of close to 200 000 g/mol.

### EXAMPLE 2: ISOBORNYL ACRYLATE/2-ETHYLHEXYL ACRYLATE/MALEIC ANHYDRIDE COPOLYMER (65/25/10 BY WEIGHT)

The polymer was prepared according to the procedure of example 1, using 65 g of isobornyl acrylate, 25 g of 2-ethylhexyl acrylate and 10 g of maleic anhydride. A solution containing 30% by weight of the copolymer in isododecane (yield of greater than 90%) was finally obtained.

The polymer obtained has a molecular weight (Mw) of close to 200 000 g/mol.

### EXAMPLE 3: ISOBORNYL ACRYLATE/2-ETHYLHEXYL ACRYLATE/MALEIC ANHYDRIDE COPOLYMER (75/20/5 BY WEIGHT)

The polymer was prepared according to the procedure of example 1, using 75 g of isobornyl acrylate, 20 g of 2-ethylhexyl acrylate and 10 g of maleic anhydride. A solution containing 30% by weight of the copolymer in isododecane (yield of greater than 90%) was finally obtained.

The polymer obtained has a molecular weight (Mw) of close to 200 000 g/mol.

### EXAMPLE 4: ISOBORNYL ACRYLATE/2-ETHYLHEXYL ACRYLATE/MALEIC ANHYDRIDE COPOLYMER (60/20/20 BY WEIGHT)

The polymer was prepared according to the procedure of example 1, using 60 g of isobornyl acrylate, 20 g of 2-ethylhexyl acrylate and 20 g of maleic anhydride. A solution containing 36% by weight of the copolymer in isododecane (yield of greater than 90%) was finally obtained.

The polymer obtained has a molecular weight (Mw) of close to 200 000 g/mol.

### EXAMPLE 5

Demonstration of the Tensioning Effect of the Polymers Used According to the Invention

This test consists in comparing, in vitro, the tensioning capacity of the polymer to be evaluated, relative to a reference tensioning polymer: Hybridur® 875 polymer dispersion from Air Products (aqueous dispersion at 40% by weight of particles of an interpenetrated network of polyurethane and acrylic polymers). The polymer to be evaluated was deposited on a nitrile rubber strip cut from a glove sold under the reference Safeskin Nitrile Criticial No. 038846 by the company Dominique Dutscher SA, having a surface area of 3.5 cm², stretched taut beforehand on a support. An aqueous solution containing the polymer to be evaluated is therefore deposited on the elastomer strip, by depositing 1.8 mg (of solids) of polymer.

26 μl of an aqueous solution containing 7% AM of Hybridur® 875 polymer were thus placed on a nitrile rubber strip so as thus to obtain a reference tensioning strip, and 26 μl of a solution containing 7% AM of acrylic polymer to be evaluated in an isododecane/ethanol mixture (70/30 weight/weight) were placed on another strip.

After drying for 24 hours at room temperature (25° C.), the curving (retraction) of the strip treated with the acrylic polymer is observed in comparison with that obtained with the control (Hybridur® 875).

The tensioning effect of the acrylic polymer was also evaluated in the presence of 3-aminopropyltriethoxysilane (APTES). The solution of acrylic polymer (at 1.75% AM) and the APTES were then mixed in the proportions indicated in the table hereinafter, and then an amount of the prepared mixture was deposited on the nitrile rubber strips.

### Polymer of Example 1

| | Proportion of | | | |
|---|---|---|---|---|
| | APTES (molar | | | |
| | equivalent) | | | Volume |
| | relative to the | Amount of | Amount of | of the |
| | anhydride | APTES | solution of the | mixture to |
| | groups of the | added | polymer of ex 1 | be taken |
| Example | polymer | (in g) | (g) | for a strip |
| 1a | 0.5 | 0.0355 | 1 | 26 μl |
| 1b | 1 | 0.0711 | 1 | 26 μl |

The tensioning effect obtained was measured according to the protocol previously described. The water resistance of the tensioning effect was then evaluated by immersing the rubber strips treated with the polymer to be evaluated in water at room temperature (25° C.) for 10 minutes, followed by evaluating the tensioning effect after 1 hour of drying.

The following results were obtained:

| | | Tensioning effect |
|---|---|---|
| | | after immersion in |
| Polymer tested | Tensioning effect | water |
| Hybridure 875 reference | correct | Correct |
| Example 1 | greater than the | greater than the |
| | reference | reference |
| Example 1a | greater than the | greater than the |
| | reference | reference |
| Example 1b | greater than the | greater than the |
| | reference | reference |

The results obtained show that the polymer of example 1 used alone forms a film which has a good tensioning effect, including after immersion in water. The tensioning performance is improved in the presence of APTES according to examples 1 a and 1 b.

### Polymer of Example 2

| | Proportion of | | | |
|---|---|---|---|---|
| | APTES (molar | | | |
| | equivalent) | | | Volume |
| | relative to the | Amount of | Amount of | of the |
| | anhydride | APTES | solution of the | mixture to |
| | groups of the | added | polymer of ex 2 | be taken |
| Example | polymer | (in g) | (g) | for a strip |
| 2a | 0.5 | 0.0261 | 1 | 26 μl |
| 2b | 1 | 0.0521 | 1 | 26 μl |
| 2c | 2 | 0.1043 | 1 | 26 μl |

The following results were obtained:

| | | Tensioning effect |
|---|---|---|
| | | after immersion in |
| Polymer tested | Tensioning effect | water |
| Hybridure 875 reference | correct | Correct |
| Example 2 | comparable to the | greater than the |
| | reference | reference |
| Example 2a | greater than the | greater than the |
| | reference | reference |
| Example 2b | greater than the | greater than the |
| | reference | reference |
| Example 2c | comparable to the | comparable to the |
| | reference | reference |

The results obtained show that the polymer of example 2 used alone forms a film which has a good tensioning effect, including after immersion in water. The tensioning performance is improved in the presence of APTES according to examples 2 a, 2 b and 2 c.

### Polymer of Example 3

| | Proportion of | | | |
|---|---|---|---|---|
| | APTES (molar | | | |
| | equivalent) | | | Volume |
| | relative to the | Amount of | Amount of | of the |
| | anhydride | APTES | solution of the | mixture to |
| | groups of the | added | polymer of ex 3 | be taken |
| Example | polymer | (in g) | (g) | for a strip |
| 3a | 0.5 | 0.0263 | 1 | 26 μl |
| 3b | 1 | 0.0526 | 1 | 26 μl |
| 3c | 2 | 0.1052 | 1 | 26 μl |

The following results were obtained:

| | | Tensioning effect |
|---|---|---|
| | | after immersion in |
| Polymer tested | Tensioning effect | water |
| Hybridure 875 reference | correct | Correct |
| Example 3 | comparable to the | greater than the |
| | reference | reference |
| Example 3a | comparable to the | greater than the |
| | reference | reference |
| Example 3b | comparable to the | greater than the |
| | reference | reference |
| Example 3c | comparable to the | greater than the |
| | reference | reference |

The results obtained show that the polymer of example 3 used alone forms a film which has a good tensioning effect, including after immersion in water. The tensioning performance, after immersion in water, is improved in the presence of APTES according to examples 3 a, 3 b and 3 c.

### EXAMPLE 6

An Anti-Wrinkle Gel Having the Following Composition is Prepared:

| polymer of example 1 as a 30% by weight solution in | 7 | g AM |
|---|---|---|
| isododecane | | |
| disteardimonium hectorite/propylene carbonate in | 3 | g |
| isododecane (bentone gel ® ISDV from Elementis) | | |
| Preservatives | qs | |
| Isododecane/ethanol (80/20 w/w) | qs 100 | g |

Just before application to the skin, 1.58 g of 3-aminopropyltriethoxysilane (APTES) are added to the gel.

A similar composition is also prepared using the polymer of example 2 or 3 or 4. The composition obtained, applied to the face, makes it possible to effectively smooth out wrinkles.

### EXAMPLES 7 to 14: COSMETIC EVALUATION OF MAKEUP COMPOSITIONS

The 8 makeup compositions of base coat and of top coat described below (examples 8, 10, 12, 14 according to the invention: top coat with APTES; examples 7, 9, 11, 13 outside the invention: topcoat without APTES) were prepared.

Each base coat composition was applied onto a skin equivalent support made of elastomer by producing a deposit with a wet thickness of 100 μm, which was left to dry at room temperature (25° C.) for 24 hours.

The top coat composition was then applied onto each dry base coat deposit by producing a deposit with a wet thickness of 100 μm, which was left to dry at room temperature (25° C.) for 24 hours.

The state of the film obtained was then observed.

The resistance of the film obtained was evaluated by separately applying 0.5 ml of olive oil and 0.5 ml of sebum; after 5 minutes of contact, the surface of the film was rubbed with cotton wool and the state of the film was then observed.

The tackiness of the film and its capacity for transferring or not transferring on touching the film with a finger were also evaluated.

The evaluation was made in the following manner:

+++: very efficient evaluated cosmetic property
++: moderately efficient evaluated cosmetic property
+: sparingly efficient evaluated cosmetic property
∘: inefficient evaluated cosmetic property

The following results were obtained:

| | | Example 8 | | Example 10 |
|---|---|---|---|---|
| | Example 7 | (invention) | Example 9 | (invention) |
| Base Coat | | | | |
| Polymer of example 4 | 20 g | 20 g | 20 g | 20 g |
| Pigmentary paste | 5 g with DC | 5 g with DC | 5 g with DC | 5 g with DC |
| containing 40% by | Red 7 | Red 7 | Red 7 | Red 7 |
| weight of pigment in | | | | |
| isododecane | | | | |
| Disteardimonium | 10 g | 10 g | 10 g | 10 g |
| hectorite | | | | |
| (Bentone Gel ISD | | | | |
| V from Elementis) | | | | |
| Isododecane | 65 g | 65 g | 45 g | 45 g |
| 2-Octyldodecanol | | | 20 g | 20 g |
| Top Coat | | | | |
| APTES | | 10 g | | 10 g |
| Isododecane | | 90 g | | 90 g |
| Appearance of the | Homogeneous | Homogeneous | Homogeneous | Homogeneous |
| film | film | film | film | film |
| Olive oil resistance | + | +++ | + | +++ |
| Sebum resistance | + | +++ | + | +++ |
| Non-tacky | +++ | +++ | +++ | +++ |
| Transfer-resistant | +++ | +++ | +++ | +++ |
| | | Example 12 | | Example 14 |
| | Example 11 | (invention) | Example 13 | (invention) |
| Base Coat | | | | |
| Polymer of example 4 | 20 g | 20 g | 20 g | 20 g |
| Pigmentary paste | 5 g with | 5 g with | 5 g with | 5 g with |
| containing 40% by | black iron | black iron | black iron | black iron |
| weight of pigment in | oxide | oxide | oxide | oxide |
| isododecane | | | | |
| Disteardimonium | 10 g | 10 g | 10 g | 10 g |
| hectorite | | | | |
| (Bentone Gel ISD | | | | |
| V from Elementis) | | | | |
| Isododecane | 65 g | 65 g | 45 g | 45 g |
| 2-Octyldodecanol | | | 20 g | 20 g |
| Top Coat | | | | |
| APTES | | 10 g | | 10 g |
| Isododecane | | 90 g | | 90 g |
| Appearance of the | Homogeneous | Homogeneous | Homogeneous | Homogeneous |
| film | film | film | film | film |
| Olive oil resistance | + | +++ | + | +++ |
| Sebum resistance | + | +++ | + | +++ |
| Non-tacky | +++ | +++ | +++ | +++ |
| Transfer-resistant | +++ | +++ | +++ | +++ |

The results obtained show that the deposits resulting from the application of polymer 4, with or without 2-octyldodecanol, followed by APTES (examples 8, 10; 12, 14) form a non-tacky homogeneous film that does not transfer to the finger, and that is resistant to oil and to sebum, whereas the application of polymer 4 alone (examples 7, 9; 11,13) forms a deposit that has poorer resistance to oil and to sebum.

Thus, the resistance of the film to contact with olive oil and sebum is markedly improved by the application of the top coat composition comprising the APTES.

### EXAMPLE 15: COSMETIC EVALUATION OF HAIR COMPOSITION

0.5 g of a base coat composition containing 10% AM of the polymer of example 1 in isododecane was applied to a 2.7 g lock of washed and dried hair (lock No. 1). The treated lock was left to dry naturally (25° C.) for 24 hours. 0.5 g of a top coat composition containing 10% AM of APTES in isododecane was then applied to the lock, which was then left to dry naturally for 24 hours.

The persistence with respect to water and the fixing property of the treated lock were then evaluated by immersing the treated lock in water for 5 minutes. The lock was then dried manually, followed by drying under a hood. The rigidity of the lock was observed by comparison with a nontreated lock.

It was found that the lock treated before and after immersion in water has a rigid form with good hair fixing. The fixing of the hair thus shows good persistence with respect to water.

### EXAMPLE 16: COSMETIC EVALUATION OF MASCARA COMPOSITION

A base coat composition containing 20% AM of polymer of example 4, 5% of red iron oxide, 10% of disteardimonium hectorite (Bentone Gel ISD V from Elementis) and 65% of isododecane was applied to a false eyelash specimen. The treated eyelashes were left to dry naturally (25° C.) for 24 hours.

0.5 g of a top coat composition containing 10% AM of APTES in isododecane was then applied to the lock, which was then left to dry naturally for 24 hours.

The persistence with respect to sebum of the deposit formed on the treated eyelashes was then evaluated by immersing the treated eyelashes in artificial sebum for 5 minutes. The eyelashes were then left to dry naturally and were rubbed on blotting paper. No trace of deposit was observed on the paper: the deposit formed on the eyelashes is thus sebum-resistant.

1. A process for caring for or making up keratin materials, comprising:
either the topical application to the keratin materials of an anhydrous (extemporaneous) mixture of a cosmetic composition comprising a maleic anhydride acrylic polymer and of an amino alkoxysilane compound (I) or of a cosmetic composition containing same;
or the sequential application to the keratin materials of an anhydrous cosmetic composition comprising a maleic anhydride acrylic polymer and of an amino alkoxysilane compound (I) or of an anhydrous cosmetic composition containing same,
said maleic anhydride acrylic polymer being able to be obtained by polymerization of:
(a) 50% to 90% by weight, relative to the total weight of monomers, of isobornyl (meth)acrylate
(b) 1% to 50% by weight of maleic anhydride
(c) 0 to 49% by weight of additional (meth)acrylate monomer chosen from:
(i) linear or branched, saturated or unsaturated C₁-C₂₀ alkyl (meth)acrylates, optionally interrupted with one or more non-adjacent heteroatoms chosen from O and S or with a group NR, R being a C₁-C₄ alkyl group, optionally substituted with a phenyl or furfuryl group;
(ii) saturated C₄-C₈ cycloalkyl (meth)acrylates optionally interrupted with O or NH;
said amino alkoxysilane having the formula (I):
R—NH—R₁Si(OR₂)_{z}(R₃)ₓ (I)
in which:
R₁ is a linear or branched, saturated or unsaturated, cyclic or acyclic C₁-C₂₀ hydrocarbon-based divalent group, which may be interrupted in its chain with a heteroatom (O, S, NH) or a carbonyl group (CO), R₁ being linked to the silicon atom directly via a carbon atom;
R=H or a C₁-C₄ alkyl group, preferably H;
R₂ and R₃, which may be identical or different, represent a linear or branched alkyl group comprising from 1 to 6 carbon atoms,
z denotes an integer ranging from 1 to 3, and
x denotes an integer ranging from 0 to 2,
with z+x=3.

2. The process as claimed in claim 1, wherein said maleic anhydride acrylic polymer is derived from the polymerization of:
(a) 50% to 80% by weight, relative to the total weight of monomers, of isobornyl (meth)acrylate
(b) 5% to 30% by weight of maleic anhydride
(c) 15% to 30% by weight of said additional (meth)acrylate monomer.

3. The process as claim 1, wherein said maleic anhydride acrylic polymer is derived from the polymerization of:
(a) 60% to 80% by weight, relative to the total weight of monomers, of isobornyl (meth)acrylate
(b) 5% to 25% by weight of maleic anhydride
(c) 15% to 30% by weight of said additional (meth)acrylate monomer.

4. The process as claimed in claim 1, wherein the maleic anhydride acrylic polymer is derived from the polymerization of:
(a) 50% to 80% by weight, relative to the total weight of monomers, of isobornyl (meth)acrylate
(b) 5% to 15% by weight of maleic anhydride
(c) 15% to 30% by weight of said additional (meth)acrylate monomer.

5. The process as claimed in claim 1, wherein the maleic anhydride acrylic polymer is derived from the polymerization of:
(a) 60% to 80% by weight, relative to the total weight of monomers, of isobornyl (meth)acrylate
(b) 5% to 12% by weight of maleic anhydride
(c) 18% to 30% by weight of said additional (meth)acrylate monomer.

6. The process as claimed in claim 1, wherein said additional (meth)acrylate monomer is chosen from C₆-C₁₆ alkyl (meth)acrylates and preferably from C₆-C₁₆ alkyl acrylates.

7. The process as claimed in claim 1, wherein said maleic anhydride acrylic polymer is derived from the polymerization of:
(a) 50% to 80% by weight, relative to the total weight of monomers, of isobornyl (meth)acrylate
(b) 5% to 30% by weight of maleic anhydride
(c) 15% to 30% by weight of C₆-C₁₆ alkyl acrylate monomer.

8. The process as claimed in claim 1, wherein the maleic anhydride acrylic polymer is derived from the polymerization of:
(a) 50% to 80% by weight, relative to the total weight of monomers, of isobornyl (meth)acrylate
(b) 5% to 30% by weight of maleic anhydride
(c) 15% to 30% by weight of C₆-C₁₆ alkyl acrylate monomer.

9. The process as claimed in claim 1, wherein the acrylic polymer comprises isobornyl acrylate, 2-ethylhexyl acrylate and maleic anhydride.

10. The process as claimed in claim 1, wherein the acrylic polymer has a weight-average molecular weight ranging from 5000 to 1 000 000 g/mol.

11. The process as claimed in claim 1, wherein, for the amino alkoxysilane (I):
R=H;
R₁ is a linear saturated C₁-C₆;
R₂ represents an alkyl group;
R₃ represents an alkyl group.

12. The process as claimed in claim 1, wherein the amino alkoxysilane (I) is chosen from 3-aminopropyltriethoxysilane, 3-aminoethyltriethoxysilane, 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane; preferably chosen from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane.

13. The process as claimed in claim 1, wherein the amino alkoxysilane (I) is used in a mole ratio of amino alkoxysilane/maleic anhydride group of the acrylic polymer ranging from 0.01 to 10.

14. The process as claimed in claim 1, wherein the acrylic polymer is present in a content ranging from 0.1% to 10% by weight, relative to the total weight of the composition.

15. The process as claimed in claim 1, wherein the composition comprises a hydrocarbon-based oil.

16. The process as claimed in claim 1, wherein a mixture, prepared less than 5 minutes before application to keratin materials, of the composition comprising the maleic anhydride acrylic polymer and of the amino alkoxysilane or of the composition containing same is applied to the keratin materials.

17. The process as claimed in claim 1, wherein the composition comprising the maleic anhydride acrylic polymer is first applied to keratin materials, and the amino alkoxysilane (I) or an anhydrous composition containing same and comprising a physiologically acceptable medium is then applied.

18. The process as claimed in claim 1, wherein the amino alkoxysilane (I), or an anhydrous composition containing same and comprising a physiologically acceptable medium, is first applied to keratin materials, and the composition comprising the maleic anhydride acrylic polymer is then applied.

19. The process as claimed in claim 1, wherein it is applied to the skin, more particularly the facial skin, in particular wrinkled skin.

20. The process as claimed in claim 1, wherein it is intended for attenuating wrinkles.

21. The cosmetic use, as a tensioning agent for the skin, of a maleic anhydride acrylic polymer
and as a mixture with an amino alkoxysilane (I), or of an anhydrous composition containing same and comprising a physiologically acceptable medium, wherein said maleic anhydride acrylic polymer and said amino alkoxysilane (I) are defined according to claim 1.

22. A composition obtained by mixing an anhydrous composition comprising a maleic anhydride acrylic polymer
and an amino alkoxysilane (I)
or an anhydrous composition containing same and comprising a physiologically acceptable medium, wherein said maleic anhydride acrylic polymer and said amino alkoxysilane (I) are defined according to claim 1.

23. The composition as claimed in claim 1, wherein it is a makeup composition comprising a volatile oil and a nonvolatile oil, preferably a hydrocarbon-based volatile oil and a hydrocarbon-based nonvolatile oil.

24. A kit comprising a first anhydrous composition comprising a maleic anhydride acrylic polymer and a second anhydrous composition comprising an amino alkoxysilane (I) wherein said maleic anhydride acrylic polymer and said amino alkoxysilane (I) are defined according to claim 1 and comprising a physiologically acceptable medium, the first and second compositions each being packaged in a separate packaging assembly.

25. A polymer that may be obtained by reacting an amino alkoxysilane (I) with a maleic anhydride acrylic polymer wherein said maleic anhydride acrylic polymer and said amino alkoxysilane (I) are defined according to claim 1.
